(19) United States
(12) Patent Application Publication
(10) Pub. No.: US 2021/0345990 A1
(43) Pub. Date: Nov. 11, 2021

(54) ULTRASOUND IMAGING APPARATUS, OPERATING METHOD OF ULTRASOUND IMAGING APPARATUS, AND COMPUTER-READABLE RECORDING MEDIUM

(71) Applicant: OLYMPUS CORPORATION, Tokyo (JP)

(72) Inventor: Hiromasa NOGUCHI, Tokyo (JP)

(73) Assignee: OLYMPUS CORPORATION, Tokyo (JP)

(21) Appl. No.: 17/380,248

(22) Filed: Jul. 20, 2021

Related U.S. Application Data
(63) Continuation of application No. PCT/JP2019/003454, filed on Jan. 31, 2019.

(51) Int. Cl. A61B 8/14; A61B 8/08; A61B 8/00

(52) U.S. Cl. CPC A61B 8/14; A61B 8/4245; A61B 8/5207

(57) ABSTRACT

An ultrasound imaging apparatus includes: a processor configured to transmit a signal for transmitting an ultrasound wave toward an observation point from an ultrasound probe, receive an echo signal that is obtained by converting an ultrasound wave into an electrical signal, generate information relating to an attenuation factor by comparing a first intensity of a first echo signal with a second intensity of a second echo signal, the first echo signal being a signal which, after being transmitted via a first path and being reflected by the observation point, has been received via the first path, the second echo signal being a signal which, after being reflected by the observation point, has been received via a second path, the second path being a path that is different from the first path and that is equal in length to the first path, and generate evaluation information representing a comparison result.

### CROSS-REFERENCE TO RELATED APPLICATION

This application is a continuation of International Application No. PCT/JP2019/003454, filed on Jan. 31, 2019, the entire contents of which are incorporated herein by reference.

### BACKGROUND

### 1. Technical Field

The present disclosure relates to an ultrasound imaging apparatus that uses ultrasound waves to observe the tissue of an observation target, an operating method of the ultrasound imaging apparatus, and a computer-readable recording medium.

### 2. Related Art

Ultrasound waves are sometimes adopted to observe the characteristics of biological tissue or a material constituting an observation target. More specifically, by transmitting ultrasound waves to an observation target and subjecting an ultrasound echo reflected by the observation target to predetermined signal processing, information relating to the characteristics of the observation target is acquired (see, for example, Japanese Patent Publication No. 2016-531713).

### SUMMARY

In some embodiments, an ultrasound imaging apparatus includes: a processor configured to transmit a signal for transmitting an ultrasound wave toward an observation point from an ultrasound probe, receive an echo signal that is obtained by converting an ultrasound wave received by the ultrasound probe into an electrical signal, generate information relating to an attenuation factor by comparing a first intensity of a first echo signal with a second intensity of a second echo signal, the first echo signal being a signal which, after being transmitted via a first path and being reflected by the observation point, has been received via the first path, the second echo signal being a signal which, after being reflected by the observation point, has been received via a second path, the second path being a path that is different from the first path and that is equal in length to the first path, and generate evaluation information representing a comparison result.

In some embodiments, provided is an operating method of an ultrasound imaging apparatus configured to generate an ultrasound image based on an ultrasound signal acquired by an ultrasound probe provided with an ultrasound transducer that transmits an ultrasound wave toward an observation target and receives an ultrasound wave reflected by the observation target. The method includes: transmitting a signal for transmitting an ultrasound wave toward an observation point from the ultrasound probe, receiving an echo signal that is obtained by converting an ultrasound wave received by the ultrasound probe into an electrical signal; generating information relating to an attenuation factor by comparing a first intensity of a first echo signal with a second intensity of a second echo signal, the first echo signal being a signal which, after being transmitted via a first path and being reflected by the observation point, has been received via the first path, the second echo signal being a signal which after being reflected by the observation point, has been received via a second path, the second path being a path that is different from the first path and that is equal in length to the first path; and generating evaluation information representing a comparison result.

In some embodiments, provided is a non-transitory computer-readable recording medium with an executable program stored thereon. The program causes an ultrasound imaging apparatus configured to generate an ultrasound image based on an ultrasound signal acquired by an ultrasound probe provided with an ultrasound transducer that transmits an ultrasound wave toward an observation target and receives an ultrasound wave reflected by the observation target, to execute: transmitting a signal for transmitting an ultrasound wave toward an observation point from the ultrasound probe, receiving an echo signal that is obtained by converting an ultrasound wave received by the ultrasound probe into an electrical signal; generating information relating to an attenuation factor by comparing a first intensity of a first echo signal with a second intensity of a second echo signal, the first echo signal being a signal which, after being transmitted via a first path and being reflected by the observation point, has been received via the first path, the second echo signal being a signal which, after being reflected by the observation point, has been received via a second path, the second path being a path that is different from the first path and that is equal in length to the first path; and generating evaluation information representing a comparison result.

In some embodiments, an ultrasound imaging apparatus includes: a processor configured to transmit a signal for transmitting an ultrasound wave toward an observation point from an ultrasound probe, receive an echo signal that is obtained by converting an ultrasound wave received by the ultrasound probe into an electrical signal, set a first region of interest including a first path and a second region of interest including a second path for data groups on scanning planes of the ultrasound probe, compare a first intensity of a first echo signal with a second intensity of a second echo signal, the first echo signal being a signal which, after being transmitted via the first path and being reflected by the observation point, has been received via the first path, the second echo signal which, after being reflected by the observation point, has been received via the second path, the second path being a path that is different from the first path and that is equal in length to the first path, and generate evaluation information representing a comparison result.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating the configuration of an ultrasound imaging system provided with an ultrasound imaging apparatus according to a first embodiment of the disclosure;

FIG. 2 is a diagram illustrating the relationship between reception depth and amplification factor in amplification processing performed by a signal amplification unit of an ultrasound imaging apparatus according to the first embodiment of the disclosure;

FIG. 3 is a diagram illustrating the relationship between reception depth and amplification factor in amplification correction processing performed by an amplification correction unit of an ultrasound imaging apparatus according to the first embodiment of the disclosure;

FIG. 4 is a diagram schematically illustrating a data array in one sound ray of an ultrasound signal;

FIG. 5 is a diagram illustrating an example of a frequency spectrum which is calculated by a frequency analysis unit of the ultrasound imaging apparatus according to the first embodiment of the disclosure;

FIG. 6 is a diagram illustrating a straight line that has, as a parameter, a correction feature that is corrected by an attenuation correction unit of the ultrasound imaging apparatus according to the first embodiment of the disclosure;

FIG. 7 is a diagram illustrating processing to calculate the relative attenuation factor which is performed by the ultrasound imaging apparatus according to the first embodiment of the disclosure;

FIG. 8 is a diagram illustrating processing to calculate the relative attenuation factor which is performed by the ultrasound imaging apparatus according to the first embodiment of the disclosure;

FIG. 9 is a diagram illustrating processing to calculate the relative attenuation factor which is performed by the ultrasound imaging apparatus according to the first embodiment of the disclosure;

FIG. 10 is a diagram illustrating processing to calculate the relative attenuation factor which is performed by the ultrasound imaging apparatus according to the first embodiment of the disclosure;

FIG. 11 is a diagram illustrating processing to calculate the relative attenuation factor which is performed by the ultrasound imaging apparatus according to the first embodiment of the disclosure;

FIG. 12 is a diagram illustrating processing to calculate the relative attenuation factor which is performed by the ultrasound imaging apparatus according to the first embodiment of the disclosure;

FIG. 13 is a diagram illustrating processing to calculate the relative attenuation factor which is performed by the ultrasound imaging apparatus according to the first embodiment of the disclosure;

FIG. 14 is a diagram illustrating processing to calculate the relative attenuation factor which is performed by the ultrasound imaging apparatus according to the first embodiment of the disclosure;

FIG. 15 is a diagram illustrating processing to calculate the relative attenuation factor which is performed by the ultrasound imaging apparatus according to the first embodiment of the disclosure;

FIG. 16 is a diagram illustrating processing to calculate the relative attenuation factor which is performed by the ultrasound imaging apparatus according to the first embodiment of the disclosure;

FIG. 17 is a diagram illustrating processing to calculate the relative attenuation factor which is performed by the ultrasound imaging apparatus according to the first embodiment of the disclosure;

FIG. 18 is a diagram illustrating processing to calculate the relative attenuation factor which is performed by the ultrasound imaging apparatus according to the first embodiment of the disclosure;

FIG. 19 is a diagram illustrating processing to calculate the relative attenuation factor which is performed by the ultrasound imaging apparatus according to the first embodiment of the disclosure;

FIG. 20 is a diagram schematically illustrating a display example of a relative attenuation factor distribution image on a display device of the ultrasound imaging apparatus according to the first embodiment of the disclosure;

FIG. 21 is a flowchart providing an overview of processing performed by the ultrasound imaging apparatus according to the first embodiment of the disclosure;

FIG. 22 is a flowchart illustrating an overview of processing executed by the frequency analysis unit of the ultrasound imaging apparatus according to the first embodiment of the disclosure;

FIG. 23 is a diagram schematically illustrating a display example of a feature image on the display device of the ultrasound imaging apparatus according to the first embodiment of the disclosure; and

FIG. 24 is a block diagram illustrating the configuration of an ultrasound imaging system provided with an ultrasound imaging apparatus according to a second embodiment of the disclosure.

### DETAILED DESCRIPTION

A mode for carrying out the disclosure (hereinafter called “the embodiment”) will be described hereinbelow with reference to the attached drawings.

### First Embodiment

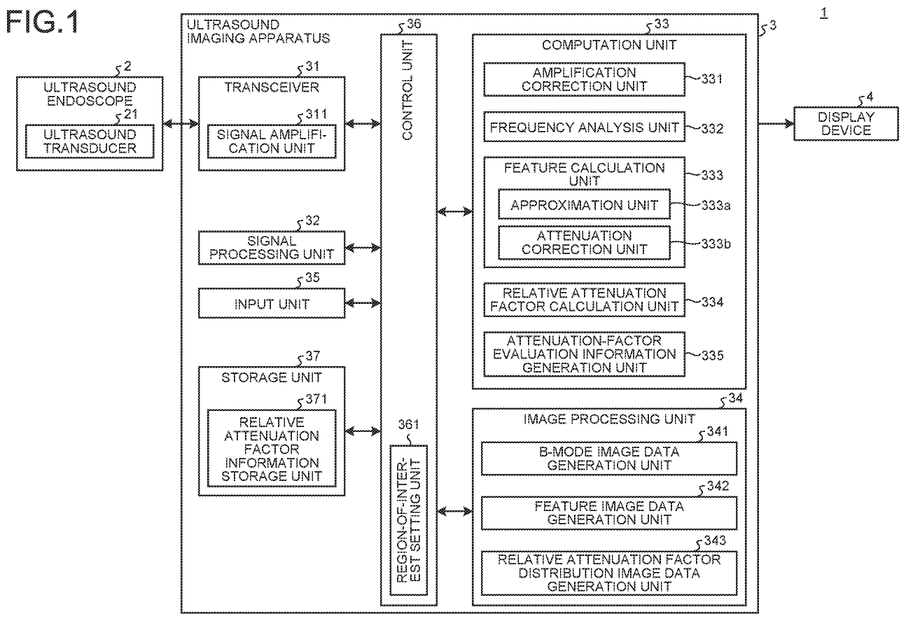

FIG. 1 is a block diagram illustrating the configuration of an ultrasound imaging system 1 provided with an ultrasound imaging apparatus 3 according to a first embodiment of the disclosure. The ultrasound imaging system 1 illustrated in FIG. 1 comprises: an ultrasound endoscope 2 (ultrasound probe) that transmits ultrasound waves to a test subject constituting an observation target and receives ultrasound waves reflected by the test subject; an ultrasound imaging apparatus 3 that generates an ultrasound image on the basis of an ultrasound signal acquired by the ultrasound endoscope 2; and a display device 4 that displays the ultrasound image generated by the ultrasound imaging apparatus 3.

The ultrasound endoscope 2 has, at the distal end thereof, an ultrasound transducer 21 that converts an electrical pulse signal received from the ultrasound imaging apparatus 3 to ultrasound pulses (acoustic pulses) and projects said pulses onto a test subject, converts an ultrasound echo reflected by the test subject into an electrical echo signal represented by a voltage variation, and outputs said echo signal. The ultrasound transducer 21 is provided with two-dimensionally arranged piezoelectric elements and transmits and receives ultrasound waves using the piezoelectric elements. The ultrasound transducer 21 may be a convex transducer, a linear transducer, or a radial transducer.

The ultrasound endoscope 2 normally has an imaging optical system and an image sensor and is capable of being inserted into the digestive tract (esophagus, stomach, duodenum, large intestine) or respiratory organs (trachea, bronchus) of the test subject to image the digestive tract, respiratory organs, and surrounding organs (pancreas, gallbladder, bile duct, biliary tract, lymph nodes, mediastinal organs, blood vessels, and so forth). The ultrasound endoscope 2 also has a light guide for guiding illumination light that is projected onto the test subject during imaging. The distal end of the light guide reaches as far as the distal end of the part of the ultrasound endoscope 2 inserted into the test subject, while the proximal end of the light guide is connected to a light source device for generating the illumination light. Note that the disclosure is not limited to the ultrasound endoscope 2, rather, an ultrasound probe without an imaging optical system or an image sensor may also be used.

The ultrasound imaging apparatus 3 is provided with: a transceiver 31 which is electrically connected to the ultrasound endoscope 2 and that transmits, to the ultrasound transducer 21, a transmission signal (pulse signal) consisting of high-voltage pulses on the basis of a predetermined waveform and transmission timing, receives an echo signal constituting an electrical reception signal from the ultrasound transducer 21, and generates and outputs digital high-frequency (RF: Radio Frequency) signal data (called RF data hereinbelow); a signal processing unit 32 that generates digital B-mode reception data on the basis of the RF data received from the transceiver 31; a computation unit 33 that performs predetermined computation with respect to the RF data received from the transceiver 31; an image processing unit 34 that generates various image data; an input unit 35 that is implemented using a user interface such as a keyboard, mouse, or touch panel and that receives inputs of various information; a control unit 36 that controls the entire ultrasound imaging system 1; and a storage unit 37 that stores various information required to operate the ultrasound imaging apparatus 3.

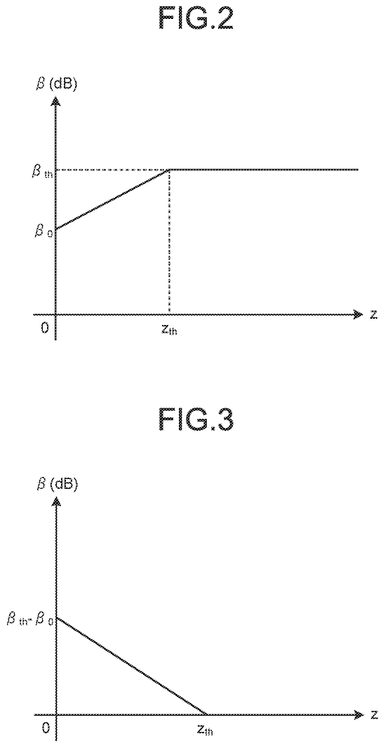

The transceiver 31 has a signal amplification unit 311 that amplifies the echo signal. The signal amplification unit 311 performs STC (Sensitivity Time Control) correction in which amplification is performed using an amplification factor that rises as the reception depth of the echo signal increases. FIG. 2 is a diagram illustrating the relationship between the reception depth and the amplification factor in the amplification processing performed by the signal amplification unit 311. The reception depth z illustrated in FIG. 2 is the amount calculated on the basis of the time elapsed since the starting point for receiving ultrasound waves. As illustrated in FIG. 2, when the reception depth z is smaller than a threshold value zₜₕ, the amplification factor β(dB) performs amplification linearly from β₀ to βₜₕ(>β₀) as the reception depth z increases. Furthermore, the amplification factor β takes a fixed value βₜₕ when the reception depth z is equal to or greater than the threshold value zₜₕ. The threshold value zₜₕ is a value such that the ultrasound signal received from the observation target is almost attenuated and the noise becomes dominant. More commonly, when the reception depth z is less than the threshold value zₜₕ, the amplification factor β may be increased monotonically as the reception depth z increases. Note that the relationship illustrated in FIG. 2 is pre-stored in the storage unit 37.

The transceiver 31 generates time-domain RF data by implementing processing such as filtering with respect to the echo signal amplified by the signal amplification unit 311 and then performing A/D conversion, and outputs said data to the signal processing unit 32 and the computation unit 33. Note that, when the ultrasound endoscope 2 has a configuration with which the ultrasound transducer 21, which is provided with a plurality of elements in an array shape, is made to perform electron scanning, the transceiver 31 has a multi-channel circuit for beam synthesis that corresponds to the plurality of elements.

The frequency band of the pulse signal transmitted by the transceiver 31 may be in a wide band that substantially covers the linear response frequency band of the electroacoustic conversion of the pulse signal of the ultrasound transducer 21 to ultrasound pulses. Furthermore, the frequency bands of the various processing of the echo signal in the signal amplification unit 311 may be in a wide band that substantially covers the linear response frequency band of the acoustic electric conversion of the ultrasound echo to the echo signal by the ultrasound transducer 21. Accordingly, when executing approximation processing of the frequency spectrum (described subsequently), a more accurate approximation can be performed.

The transceiver 31 also has functions for transmitting various control signals outputted by the control unit 36 to the ultrasound endoscope 2 and for receiving various information including an identification ID from the ultrasound endoscope 2 and transmitting this information to the control unit 36.

The signal processing unit 32 subjects the RF data to well-known processing such as a bandpass filter, envelope detection, and logarithmic transformation, and generates digital B-mode reception data. In logarithmic conversion, the normal logarithm of the amount of RF data divided by the reference voltage V_{c} is taken and expressed using decibel values. The signal processing unit 32 outputs the generated B-mode reception data to the image processing unit 34. The signal processing unit 32 is realized using a central processing unit (CPU) and various computation circuits, and the like.

The computation unit 33 includes an amplification correction unit 331, which subjects the RF data generated by the transceiver 31 to amplification correction so that the amplification factor β remains constant irrespective of the reception depth z; a frequency analysis unit 332, which performs frequency analysis by applying a fast Fourier transform (FFT) to the amplification-corrected RF data, thereby calculating a frequency spectrum; a feature calculation unit 333, which calculates a feature of the frequency spectrum on the basis of the frequency spectrum calculated by the frequency analysis unit 332; a relative attenuation factor calculation unit 334, which calculates a relative attenuation factor for evaluating the attenuation factor; and an attenuation-factor evaluation information generation unit 335, which generates evaluation information for evaluating the attenuation factor of each region of interest. The computation unit 33 is realized using a CPU and various computation circuits, and the like.

FIG. 3 is a diagram illustrating the relationship between the reception depth and the amplification factor in the amplification correction processing performed by the amplification correction unit 331. As illustrated in FIG. 3, the amplification factor β(dB) in the amplification correction processing performed by the amplification correction unit 331 takes a maximum value βₜₕ-β₀ when the reception depth z is zero, decreases linearly from a reception depth z of zero up to the threshold value zₜₕ, and is zero when the reception depth z is equal to or greater than the threshold value zₜₕ. The amplification correction unit 331 amplifies and corrects the digital RF signal according to the amplification factor determined in this way, thereby offsetting the effect of STC correction in the signal amplification unit 311 and enabling a signal with a constant amplification factor βₜₕ to be outputted. Note that the relationship between the reception depth z and the amplification factor β, as implemented by the amplification correction unit 331, is naturally different depending on the relationship between the reception depth and the amplification factor in the signal amplification unit 311.

Reasons for such amplification correction are described below. STC correction is correction processing that eliminates the effect of attenuation from the amplitude of an analog signal waveform by amplifying the amplitude of the analog signal waveform uniformly over the entire frequency range and using a monotonically increasing amplification factor for depth. Therefore, when generating a B-mode image, which is displayed by converting the amplitude of an echo signal to luminance, and when scanning uniform tissue, the luminance values will be made constant irrespective of depth by performing STC correction. That is, the effect of eliminating the effect of attenuation from the luminance values of the B-mode image can be attained.

Meanwhile, when using the results of calculating and analyzing the frequency spectrum of ultrasound waves as per the present embodiment, even STC correction does not accurately eliminate the effect of the attenuation caused by the propagation of ultrasound waves. This is because, although the attenuation amount generally varies with frequency (see Equation (1) below), the amplification factor of STC correction varies only with distance and is not frequency-dependent.

To solve the above-mentioned problem, namely that when using the results of calculating and analyzing the frequency spectrum of ultrasound waves, even STC correction does not accurately eliminate the effect of attenuation caused by the propagation of ultrasound waves, consideration may be given to outputting an STC-corrected reception signal when generating a B-mode image, but, when generating a frequency spectrum-based image, performing a new transmission which is different from the transmission to generate the B-mode image, and outputting a reception signal which has not been STC-corrected. In this case, however, the frame rate of the image data generated on the basis of the reception signal is reduced.

Therefore, in the present embodiment, the amplification factor is corrected by the amplification correction unit 331 in order to eliminate the effect of STC correction on a signal which has undergone STC correction for a B-mode image while maintaining the frame rate of the generated image data.

The frequency analysis unit 332 samples the RF data (line data) of each sound ray that has been amplification-corrected by the amplification correction unit 331 at predetermined time intervals and generates sample data. The frequency analysis unit 332 calculates the frequency spectrum at a plurality of locations (data positions) of the RF data by applying FFT processing to a sample data group. The “frequency spectrum” here means the “frequency distribution of the intensity at a certain reception depth z” obtained by applying FFT processing to the sample data group. Furthermore, the term “intensity” here refers, for example, to parameters such as the voltage of the echo signal, the power of the echo signal, the sound pressure of the ultrasound echo, the acoustic energy of the ultrasound echo, and to any of the amplitudes or time-integrated values of these parameters or combinations thereof.

In general, when the observation target is biological tissue, the frequency spectrum tends to vary depending on the properties of the biological tissue that is scanned using ultrasound waves. This is because the frequency spectrum is correlated with the size, number density, acoustic impedance, and the like, of the scattering bodies that scatter the ultrasound waves. The “properties of the biological tissue” referred to here are, for example, malignant tumors (cancer), benign tumors, endocrine tumors, mucinous tumors, normal tissues, cysts, vascular vessels, and so forth.

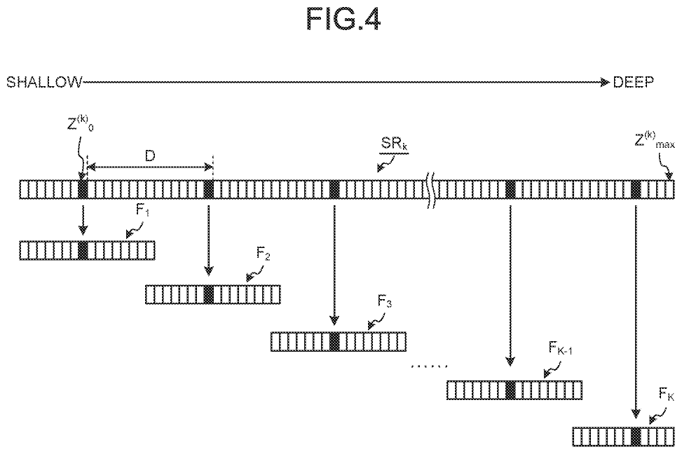

FIG. 4 is a diagram schematically illustrating a data array in one sound ray of an ultrasound signal. In the sound ray SRₖ illustrated in FIG. 4, a white or black rectangle denotes data at one sample point. In the sound ray SRₖ, the closer to the right that data is located, the further the sample data is from a deep point when measured along the sound ray SRₖ from the ultrasound transducer 21 (see the arrow in FIG. 4). The sound ray SRₖ is discretized at time intervals corresponding to the sampling frequency (50 MHz, for example) in the A/D conversion performed by the transceiver 31. FIG. 4 illustrates a case where the eighth data position of the sound ray SRₖ with number k is set as the initial value Z^{(k)}₀ in the direction of the reception depth z. However, the position of the initial value can be optionally set. The results of the calculation by the frequency analysis unit 332 are obtained as complex numbers and stored in the storage unit 37.

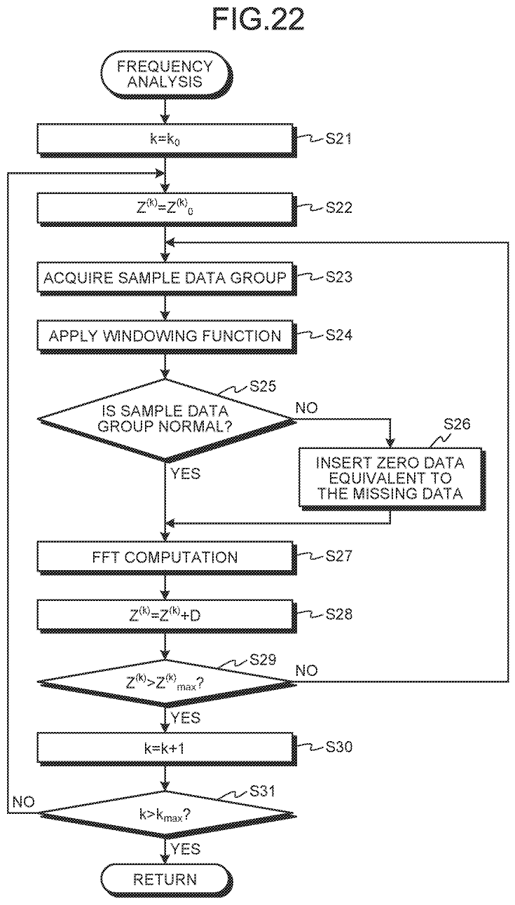

The data group Fⱼ (j=1, 2, . . . , K) illustrated in FIG. 4 is the sample data group to be subjected to FFT processing. In general, in order to perform FFT processing, the sample data group must have a data count to the power of two. In that sense, the sample data group Fⱼ (j=1, 2, . . . , K−1) is a normal data group with a data count of 16 (=2⁴), while the sample data group F_{K} is an abnormal data group due to the data count of 12. When performing FFT processing on an abnormal data group, the process of generating a normal sample data group is performed by inserting zero data equivalent to the missing amount. This point will be discussed in detail when describing the processing by the frequency analysis unit 332 (see FIG. 22).

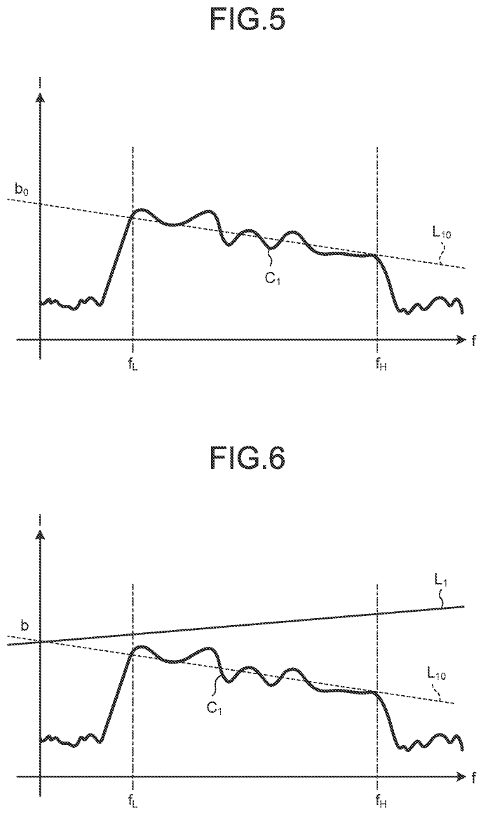

FIG. 5 is a diagram illustrating an example of a frequency spectrum which is calculated by the frequency analysis unit 332. In FIG. 5, the horizontal axis represents the frequency f. In addition, in FIG. 5, the vertical axis represents a normal logarithm (decibel representation) I=10 log₁₀(I₀/I_{c}) of an amount obtained by dividing the intensity I₀ by a reference intensity I_{c} (constant). The straight line L₁₀ illustrated in FIG. 5 (hereinafter also referred to as the regression line L₁₀) will be discussed subsequently. Note that, in the present embodiment, curves and lines are composed of a set of discrete points.

In the frequency spectrum C₁ illustrated in FIG. 5, the lower limit frequency f_{L} and upper limit frequency f_{H} of the frequency band used in subsequent computation are parameters which are determined on the basis of the frequency band of the ultrasound transducer 21, the frequency band of the pulse signal transmitted by the transceiver 31, and the like. The frequency band determined by the lower limit frequency f_{L} and the upper limit frequency f_{H} is referred to as “frequency band F” in FIG. 5 below.

The feature calculation unit 333 calculates features of a plurality of frequency spectra, respectively, within a set region of interest (hereinafter sometimes referred to as ROI (Region of Interest)). In this first embodiment, it is assumed that two regions of interest with mutually different regions have been set. The feature calculation unit 333 includes: an approximation unit 333a that calculates the feature of the frequency spectrum before the attenuation correction processing (hereinafter referred to as “pre-correction feature”) by linearly approximating the frequency spectrum; and an attenuation correction unit 333b that calculates the feature by performing attenuation correction on the pre-correction feature calculated by the approximation unit 333a.

The approximation unit 333a performs a regression analysis of the frequency spectrum in a predetermined frequency band and approximates the frequency spectrum with a linear equation (regression line), thereby calculating the pre-correction feature that characterizes this approximated linear equation. For example, in the case of the frequency spectrum C₁ illustrated in FIG. 5, the approximation unit 333a obtains the regression line L₁₀ by approximating the frequency spectrum C₁ using a linear equation through regression analysis in the frequency band F. In other words, the approximation unit 333a calculates the slope a₀ of the regression line L₁₀, the intercept b₀, and the mid-band fit c₀=a₀f_{M}+b₀, which is the value on the regression line of the center frequency f_{M}=(f_{L}+f_{H})/2 of the frequency band F, as the pre-correction feature.

Among the three pre-correction features, the slope a₀ is correlated with the size of the scattering bodies of the ultrasound waves, and in general, it is considered that the larger the scattering bodies, the smaller the slope value. Further, the intercept b₀ is correlated with the size of the scattering bodies, the difference in acoustic impedance, and the number density (concentration) of the scattering bodies. Specifically, the intercept b₀ is considered to have a larger value for larger scattering bodies, a larger value for larger differences in acoustic impedance, and a larger value for larger number densities of scattering bodies. The midband fit c₀ is an indirect parameter derived from the slope a₀ and the intercept b₀, and gives the intensity of the spectrum at the center within the effective frequency band. Therefore, the midband fit c₀ is considered to have some correlation with the luminance of the B-mode image, in addition to the size of the scattering bodies, the difference in acoustic impedance, and the number density of the scattering bodies. Note that the feature calculation unit 333 may approximate the frequency spectrum with a polynomial of the second order or higher by means of regression analysis.

The corrections made by the attenuation correction unit 333b will now be described. In general, the ultrasound wave attenuation amount A(f,z), is the attenuation that occurs during the round trip of the ultrasound wave between a reception depth 0 and the reception depth z, and is defined as the change in intensity (difference in decibel representation) before and after the round trip. It is empirically known that the attenuation amount A(f,z) is proportional to the frequency in uniform tissue and is represented by the following equation (1).

A(f,z)=2αzf (1)

Here, the proportionality constant α is a quantity called the attenuation factor. z is the reception depth of the ultrasound waves, and f is the frequency. The specific value of the attenuation factor α is determined according to the part of the body when the observation target is a living body. The unit of the attenuation factor α is, for example, dB/cm/MHz. Note that, in the present embodiment, a configuration in which the value of the attenuation factor α can be changed by an input from the input unit 35 is also possible.

The attenuation correction unit 333b calculates the features a, b, and c by performing attenuation correction on the pre-correction features (slope a₀, intercept b₀, midband fit c₀) extracted by the approximation unit 333a according to the equations (2) to (4) indicated below.

a=a₀+2αz (2)

b=b₀ (3)

c=c₀+A(f_{M},z)=c₀+2αzf_{M}(=af_{M}+b) (4)

As is clear from Equations (2) and (4), the attenuation correction unit 333b performs correction with a larger correction amount as the reception depth z of the ultrasound waves increases. Also, according to Equation (3), the correction pertaining to the intercept is a constant transformation. This is because the intercept is the frequency component corresponding to the frequency 0 (Hz) and is not affected by attenuation.

FIG. 6 is a diagram illustrating a straight line for which the features a, b, and c, which are calculated by the attenuation correction unit 333b, serve as parameters. The equation of the straight line L₁ is represented by:

I=af+b=(a₀+2αz)f+b₀ (5).

As is clear from this Equation (5), the straight line L₁ has a larger slope (a>a₀) and the same intercept (b=b₀) compared to the straight line L₁₀ before attenuation correction.

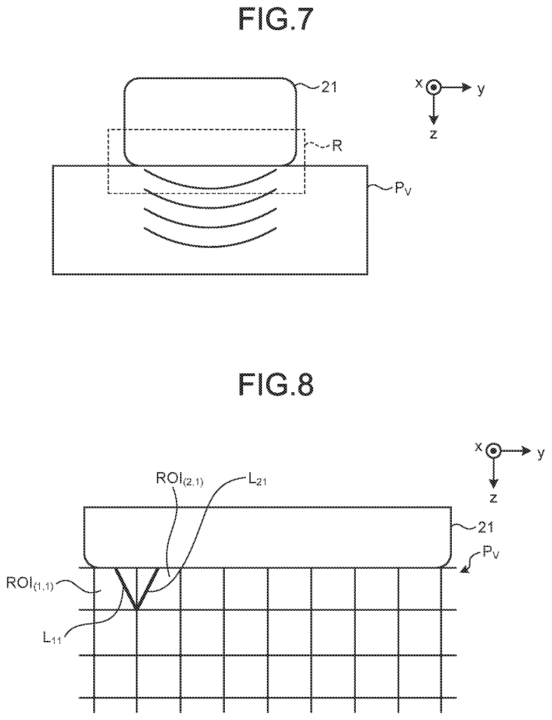

Using the reception data obtained through ultrasound waves transmission for the relative attenuation factor calculation, the relative attenuation factor calculation unit 334 calculates the relative attenuation factor by comparing the intensity of the reception echoes of mutually different paths from the same point of the test subject. Here, the relative attenuation factor is calculated using the reception data acquired by transmitting the ultrasound waves for calculating the relative attenuation factor, which is different from the B-mode reception data. The relative attenuation factor calculation unit 334 corresponds to the comparison unit. FIGS. 7 to 18 are diagrams illustrating processing to calculate the relative attenuation factor which is performed by the ultrasound imaging apparatus according to the first embodiment of the disclosure. FIG. 8 is an enlarged view of the region R illustrated in FIG. 7. The ultrasound transducer 21 will be described hereinbelow as transmitting and receiving ultrasound waves in the z direction illustrated in FIG. 7. This z direction corresponds to the aforementioned depth z. Note that the scanning plane P_{V} of the ultrasound transducer 21 is orthogonal to the x direction and parallel to the yz plane. The ultrasound transducer 21 receives ultrasound echoes from the test subject while moving the scanning plane P_{V} in the x direction.

Here, consideration is given to two paths (first and second paths), in a plurality of divided regions of interest, that the reflected echo takes when the ultrasound waves are reflected at a point (observation point) on the boundary of adjacent regions of interest. These two paths share the same observation point where ultrasound waves are reflected, and their path lengths are equal to each other. The reception strengths of the paths are denoted as G(1) and G(2), as follows: G(1) and G(2) are measured individually by transmitting and receiving ultrasound waves for each path.

G(1): Echo intensity obtained when transmitting and receiving ultrasound waves by means of the first path (dB)

G(2): Echo intensity (dB) obtained when transmitting and receiving ultrasound waves by means of the second path.

When the intensities of the transmission waves in these two paths are equal, the difference between G(1) and G(2) depends only on the difference in the attenuation factor in each region of interest. That is, the difference between G(1) and G(2) in this case does not depend on the spatial distribution of the reflectance of the test subject within each region of interest.

In FIG. 8, the divided regions of interest are assigned positional coordinates starting from a top-left position. The coordinates correspond to the coordinate (y, z), which is represented by the position in the y direction and the position in the z direction. For example, the top-left region of interest is (1,1) (denoted as ROI_{(1,1)} in FIG. 8), and the region of interest adjacent to this region of interest (1,1) in the y-direction is (2,1) (denoted as ROI_{(2,1)} in FIG. 8). Paths to the ultrasound transducer 21 from the point on the boundary of the region of interest (1,1) and of the region of interest (2,1) are the paths L₁₁ and L₂₁, respectively. For example, the path L₁₁ corresponds to the first path, and the path L₂₁ corresponds to the second path. These paths L₁₁ and L₂₁ have equal path lengths (the path length is denoted L). Since the ultrasound waves through each point are transmitted from the same ultrasound transducer 21, the intensity of the transmission waves can be considered to be equal.

When the attenuation factor of the region of interest (1,1) is d₁ and the attenuation factor of the region of interest (2,1) is d₂, the relative attenuation factor d_{1,2} of the region of interest (2,1) to the region of interest (1,1) is represented by the following equation (6).

d_{1,2}=d₂−d₁ (6)

Here, the relative attenuation factor d_{1,2} can be represented by the following equation (7).

G(1)−G(2)=2L*d₂−2L*d₁⇒d_{1,2}=(G(1)−G(2))/2L (7)

Since the path length L of pathways L₁₁ and L₂₁ is determined by the size of the region of interest, the relative attenuation factor d_{1,2} can be calculated.

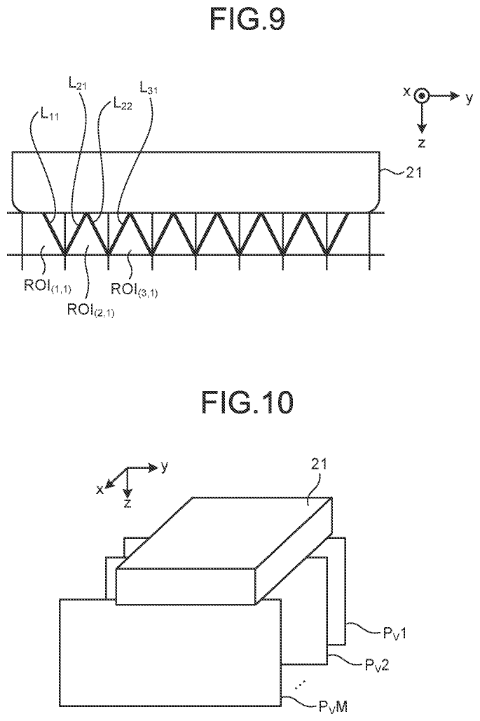

FIG. 9 is an enlarged view of the region R illustrated in FIG. 7. The relative attenuation factor calculation unit 334 uses the above equations (6) and (7) to calculate the relative attenuation factor for each region of interest. The relative attenuation factor calculation unit 334 calculates the relative attenuation factor of each region of interest with respect to the region of interest (1,1) by the same procedure as the relative attenuation factor of the region of interest ROI (2,1) to the region of interest ROI (1,1). Specifically, the relative attenuation factor d_{1,3} of the region of interest (3,1) to the region of interest (1,1) is represented by the following equation (8).

d_{1,3}=d_{1,2}+d_{2,3} (8)

Expressed generally, this equation becomes:

d_{1,n}=d_{1,2}+d_{2,3}+ . . . +d_{n-1,n} (9)

Note that n is a natural number and corresponds here to the number of regions of interest in the y-direction. The relative attenuation factor calculation unit 334 calculates the relative attenuation factor for the region of interest (1,1) for each region of interest aligned in the y-direction according to the above equation (9).

The ultrasound transducer 21, in which piezoelectric elements are two-dimensionally arranged, enables scanning in three-dimensional space. With the ultrasound transducer 21, by moving the scanning plane P_{V} (see FIG. 7) in the x-direction, it is possible to acquire ultrasound echoes for a plurality of scanning planes (scanning planes P_{V}1, P_{V}2, . . . , P_{V}M (M is a natural number) (see FIG. 10)) aligned in the x-direction. These scanning planes are, for example, perpendicular to the ultrasound-wave transmission-reception plane of the ultrasound transducer 21 and parallel to each other.

The relative attenuation factor calculation unit 334 calculates the relative attenuation factor in each scanning plane. The relative attenuation factor calculation unit 334 uses the above equations (6) to (9) to calculate, in each scanning plane, the relative attenuation factor of each region of interest aligned in the y-direction with respect to the region of interest ROI(1,1).

The relative attenuation factor calculated for each scanning plane is based on a different region of interest to be used as a reference. Specifically, the relative attenuation factor for a scanning plane P_{V}1 is calculated by using the region of interest (1,1) in this scanning plane P_{V}1 as a reference, and the relative attenuation factor for a scanning plane P_{V}2 is calculated by using the region of interest (1,1) in this scanning plane P_{V}2 as a reference.

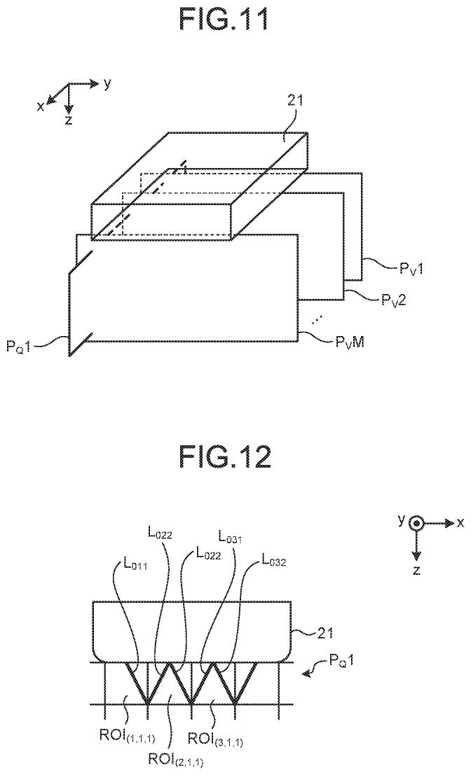

Then, we consider a plane P_{Q}1 (see FIGS. 11 and 12), which is orthogonal to the scanning planes P_{V}1, P_{V}2, . . . , P_{V}M and contains the region of interest (1,1) in each scanning plane. Hereinbelow, the region of interest (1,1) in the scanning plane P_{V}1 is represented as (1,1,1), and similarly, the region of interest (1,1) in the scanning plane P_{V}2 is represented as (2,1,1), . . . , and the region of interest (1,1) in the scanning plane P_{V}M is represented as (M,1,1). The relative attenuation factor of the region of interest (2,1,1) to the region of interest (1,1,1) is represented by d_{(1,1,1),(2,1,1)}. Further, the relative attenuation factor of the region of interest (3,1,1) to the region of interest (1,1,1) is d_{(1,1,1),(3,1,1)}, and the relative attenuation factor of the region of interest (M,1,1) to the region of interest (1,1,1) is d_{(1,1,1),(M,1,1)}. In this case, the path length of the path L₀₁₁ in the region of interest (1,1,1) is the same as the path length of the path L₀₂₁ in the region of interest (2,1,1). Similarly, the path length of the path L₀₂₂ in the region of interest (2,1,1) is the same as the path length of the path L₀₃₁ in the region of interest (3,1,1).

Equation (10) below is derived from Equation (8) above.

d_{a,b}+d_{b,c}=d_{a,c} (10)

From the above equation (10), the relative attenuation factor for which the region of interest (1,1) (region of interest (1,1,1)) on the scanning plane P_{V}1 is used as the reference can be obtained by adding the relative attenuation factor on the plane P_{Q}1 to the relative attenuation factor for each of the scanning planes P_{V}2 to P_{V}M. For example, in the plane P_{V}2,

d_{(1,1,1),(2,1,1)}+d_{(2,1,1),(2,1,2)}=d_{(1,1,1),(2,1,2)},

d_{(1,1,1),(2,1,1)}+d_{(2,1,1),(2,1,3)}=d_{(1,1,1),(2,1,3)},

d_{(1,1,1),(2,1,1)}+d_{(2,1,1),(2,1,n)}=d_{(1,1,1),(2,1,n)}.

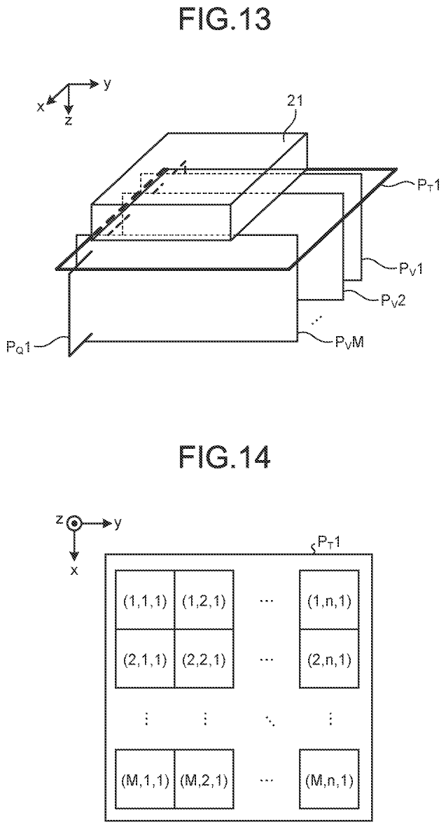

In light of the foregoing description, it is possible to calculate the relative attenuation factor for which the region of interest (1,1,1) is used as the reference, for the plane P_{T}1 (see FIG. 13) which is located at the shallowest depth from the ultrasound transducer 21. The plane P_{T}1 is parallel to the xy-plane. The plane P_{T}1 has the same shape as a plane containing a set of points equidistant from the ultrasound-wave transmission-reception plane of the ultrasound transducer 21. Located on plane P_{T}1 are: regions of interest of scanning plane P_{V}1, namely, a region of interest (1,1,1), a region of interest (1,2,1), . . . , and a region of interest (1,n,1); regions of interest (2,1,1) of scanning plane P_{V}2, namely, a region of interest (2,2,1), . . . , and a region of interest (2,n,1), . . . ; and regions of interest (M,1,1) of scanning plane P_{V}M, namely, a region of interest (M,2,1), . . . , and a region of interest (M,n,1) (see FIG. 14).

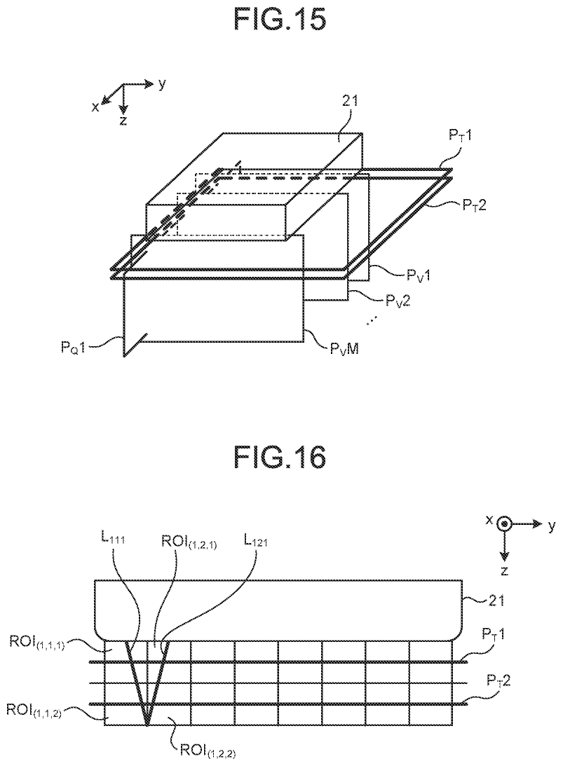

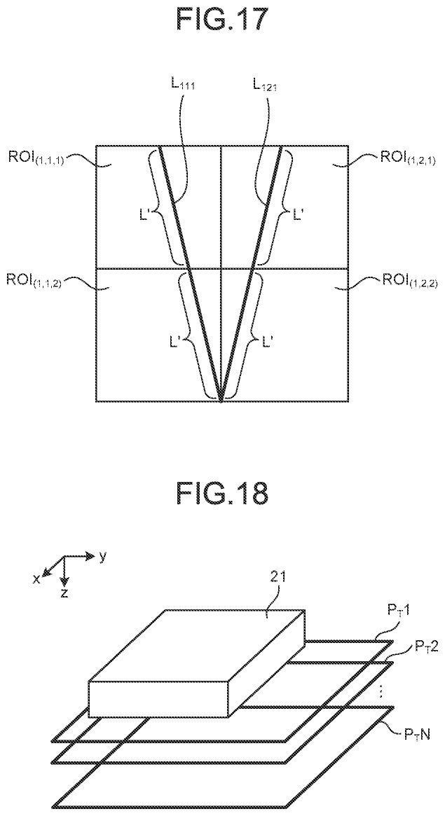

Next, consider a plane with a greater depth than plane P_{T}1 (plane P_{T}2: see FIGS. 15 and 16). First, as above, in the plane P_{T}2, paths (the third and fourth paths) are set starting from a common point through mutually different regions of interest. The reception strengths of the paths are denoted as G′(1) and G′(2), as follows: For example, in FIGS. 16 and 17, the path L₁₁₁ corresponds to the third path, and the path L₁₂₁ corresponds to the fourth path.

G′(1): Echo intensity (dB) obtained when transmitting and receiving ultrasound waves by means of the third path.

G′(2): Echo intensity (dB) obtained when transmitting and receiving ultrasound waves by means of the fourth path.

Here, the attenuation factor of the region of interest (1,1,1) on the plane P_{T}1 is d_{1,1,1}, the attenuation factor of the region of interest (1,2,1) is d_{1,2,1}, the attenuation factor of the region of interest (1,1,2) on the plane P_{T}2 is d_{1,1,2}, and the attenuation factor of the region of interest (1,2,2) is g_{1,2,2}.

The relative attenuation factor of the region of interest (1,2,2) with respect to the region of interest (1,2,1) is calculated from Equations (6) and (7) using Equation (11) below. Note that, in the path L₁₁₁ and the path L₁₂₁, the path length through one region of interest is L′. This path length L′ is also determined according to the size of the region of interest.

G′(1)−G′(2)=(2L′*d_{1,2,1}+2L′*d_{1,2,2})−(2L′*d_{1,1,1}+2L′*d_{1,1,2})=2L′(d_{1,2,1}−d_{1,1,1})+2L′(d_{1,2,2}−d_{1,1,2})=2L′*d_{(1,1,1),(1,2,1)}+2L′*d_{(1,1,2),(1,2,2)}⇒d_{(1,1,2),(1,2,2)}=(G′(1)=G′(2))/2L′−d_{(1,1,1),(1,2,1)} (11)

The above equation (11) makes it possible to calculate the relative attenuation factor d_{(1,1,2),(1,2,2)} of the region of interest (1,2,2) relative to the region of interest (1,1,2) on the plane P_{T}2. Thereupon, since the difference in attenuation factor within the region of interest is very small, the attenuation factor is assumed to be constant within the region of interest.

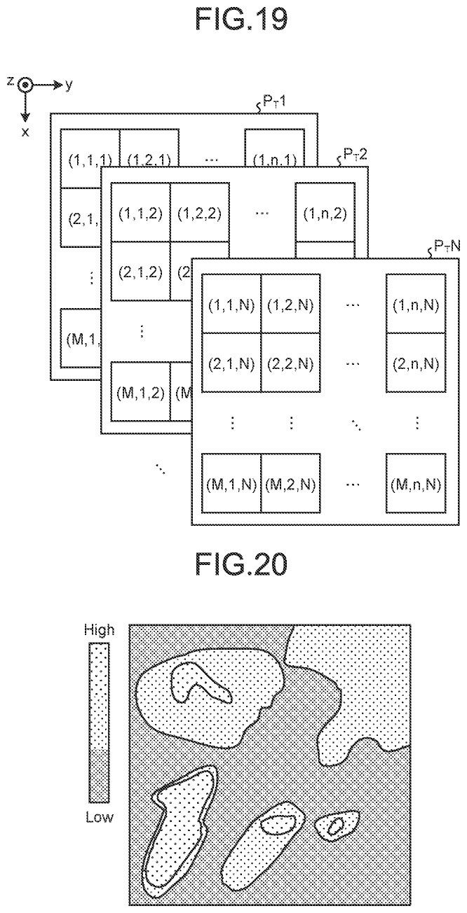

The relative attenuation factor of planes at other depths (for example, the plane P_{T}N (N is a natural number) illustrated in FIG. 18) can be calculated as per the calculation of the relative attenuation factor on the plane P_{T}2 described above. In addition to the relative attenuation factor of the plane P_{T}1 illustrated in FIG. 14, the relative attenuation factors of the planes P_{T}2, . . . , and P_{T}N are obtained (see FIG. 19). These planes P_{T}2, . . . , and P_{T}N are parallel to the plane P_{T}1 and have mutually different distances from the ultrasound-wave transmission-reception plane of the ultrasound transducer 21. Note that, when the ultrasound transducer 21 is a convex transducer or a radial transducer, the planes P_{T}1, P_{T}2, . . . , and P_{T}N form a curved surface.

The relative attenuation factor calculation unit 334 stores the calculated relative attenuation factor in the storage unit 37.

The attenuation-factor evaluation information generation unit 335 generates evaluation information for evaluating the attenuation factor of each region of interest on the basis of the relative attenuation factor distribution calculated by the relative attenuation factor calculation unit 334. The attenuation-factor evaluation information generation unit 335 uses the calculated relative attenuation factor to calculate the distribution data of the relative attenuation factor, and the statistics thereof, said data representing the distribution of the relative attenuation factors in a designated plane (for example, any of the planes P_{T}1, P_{T}2, . . . , and P_{T}N). The statistics include variance, kurtosis, skewness, and the like.

The image processing unit 34 has a B-mode image data generation unit 341 that generates B-mode image data, which is an ultrasound image to be displayed by converting the amplitude of the echo signal into luminance, a feature image data generation unit 342 that generates feature image data to be displayed together with the B-mode image by associating the feature calculated by the attenuation correction unit 333b with visual information, and a relative attenuation factor distribution image data generation unit 343 that generates relative attenuation factor distribution image data on the basis of the information generated by the attenuation-factor evaluation information generation unit 335.

The B-mode image data generation unit 341 generates B-mode image data by performing signal processing using known techniques such as gain processing, contrast processing, and gamma correction processing on the B-mode reception data received from the signal processing unit 32, as well as data thinning, or the like, which corresponds to the data step width determined according to the display range of the image on the display device 4. A B-mode image is a grayscale image in which the values of R (red), G (green), and B (blue), which are variables when the RGB color system is adopted as the color space, are matched.

The B-mode image data generation unit 341 applies a coordinate transformation to the B-mode reception data from the signal processing unit 32 to rearrange the scanning range to enable same to be represented spatially in a correct manner, and then fills in the gaps between the B-mode reception data by applying interpolation processing between the B-mode reception data, to generate B-mode image data. The B-mode image data generation unit 341 outputs the generated B-mode image data to the feature image data generation unit 342.

The feature image data generation unit 342 generates feature image data by superimposing visual information relating to the feature calculated by the feature calculation unit 333 on each pixel of the image in the B-mode image data. The feature image data generation unit 342 assigns, for example, visual information, which corresponds to the feature of the frequency spectrum calculated from one sample data group Fⱼ (j=1, 2, . . . , K) as illustrated in FIG. 4, to the pixel region corresponding to the amount of data in the sample data group Fⱼ. The feature image data generation unit 342 generates feature images, for example, by mapping a hue as visual information to one of the aforementioned slope, intercept, or midband fit. In addition to the hue, visual information related to the feature may include, for example, saturation, lightness, luminance values, and color space variables that make up a predetermined color system, such as R (red), G (green), and B (blue).

Note that, when the feature image data generation unit 342 performs gain adjustment or contrast processing, this unit 342 may adjust visual information (luminance values) independently of the gain adjustment performed by the B-mode image data generation unit 341, or may adjust the luminance difference independently of the contrast of the B-mode image data. The feature image data generation unit 342 may also be configured to set an adjustment value for each model of ultrasound endoscope 2.

Furthermore, when the feature image data generation unit 342 performs γ correction, this unit 342 may use the same correction table as the correction table for γ correction performed by the B-mode image data generation unit 341, or may use a different correction table. The feature image data generation unit 342 may also be configured to enable adjustment of the curvature of the γ curve for γ correction and the ratio between input and output for each model of ultrasound endoscope 2.

The relative attenuation factor distribution image data generation unit 343 images the distribution of relative attenuation factors on the basis of the information generated by the attenuation-factor evaluation information generation unit 335. FIG. 20 is a diagram schematically illustrating a display example of a relative attenuation factor distribution image on a display device of the ultrasound imaging apparatus according to the first embodiment of the disclosure. The relative attenuation factor distribution image data generation unit 343 generates the relative attenuation factor distribution image data by assigning preset colors (indicated by hatching in FIG. 20) to each region of interest according to the range of relative attenuation factor values.

The control unit 36 is realized using a CPU and various computation circuits, or the like, that have computation and control functions. The control unit 36 reads the information memorized and stored by the storage unit 37 from the storage unit 37, and centrally controls the ultrasound imaging apparatus 3 by executing various computation processing related to the operating method of the ultrasound imaging apparatus 3. Note that it is also possible to configure the control unit 36 using a CPU, or the like, which is common to the signal processing unit 32 and the computation unit 33.

The control unit 36 has a region-of-interest setting unit 361 that sets a region of interest for a data group according to preset conditions or instruction inputs accepted by the input unit 35. This data group corresponds to the scanning plane of the ultrasound transducer 21. In other words, the data group is a set of points (data) acquired from each position of the scanning plane, and each point in the set is located on a predetermined plane that corresponds to the scanning plane.

The region-of-interest setting unit 361 sets the region of interest (see FIG. 8) for calculating the relative attenuation factor according to the preset conditions. For example, the size of the region of interest is set according to the size of the pixel. Note that the size of the region of interest may also be set by a surgeon or other user by using the input unit 35.

The region-of-interest setting unit 361 also sets the region of interest for calculating the foregoing feature on the basis of setting inputs (instruction points) which are inputted via the input unit 35, for example. The region-of-interest setting unit 361 may be configured to arrange a frame of a preset shape on the basis of the positions of the instruction points, or may be configured to form a frame by connecting point groups of a plurality of input points.

The storage unit 37 stores the plurality of features calculated for each frequency spectrum by the attenuation correction unit 333b and the image data generated by the image processing unit 34. The storage unit 37 also has a relative attenuation factor information storage unit 371 that stores the calculated relative attenuation factor and setting conditions for the colors used for imaging.

In addition to the foregoing, the storage unit 37 stores, for example, information required for amplification processing (the relationship between amplification factor and reception depth illustrated in FIG. 2), information required for amplification correction processing (the relationship between amplification factor and reception depth illustrated in FIG. 3), information required for attenuation correction processing (see Equation (1)), and information on windowing functions required for frequency analysis processing (Hamming, Hanning, Blackman, and the like).

Furthermore, the storage unit 37 stores various programs, including an operating program for executing the operating method of the ultrasound imaging apparatus 3. The operating program can also be recorded, for wide distribution, on computer-readable recording media such as hard disks, flash memories, CD-ROMs, DVD-ROMs, and flexible disks. Note that the aforementioned various programs can also be acquired by being downloaded via a communication network. The communication network referred to here is realized, for example, by existing public line networks, a LAN (Local Area Network), a WAN (Wide Area Network), or the like, and can be wired or wireless.

The storage unit 37 with the foregoing configuration is realized a using ROM (Read Only Memory) in which various programs, or the like, are pre-installed, and a RAM (Random Access Memory), or the like, which stores computation parameters, data, and the like, for each process.

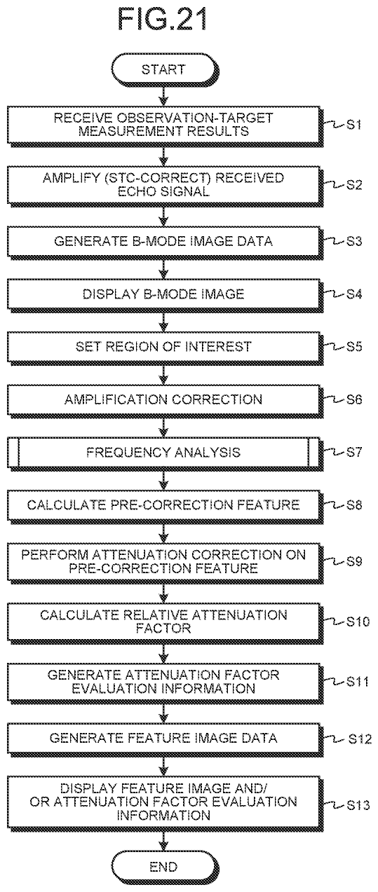

FIG. 21 is a flowchart illustrating an overview of processing performed by the ultrasound imaging apparatus 3 with the foregoing configuration. First, the ultrasound imaging apparatus 3 receives an echo signal from the ultrasound endoscope 2 as a result of measurement of the observation target by the ultrasound transducer 21 (step S1).

After receiving the echo signal from the ultrasound transducer 21, the signal amplification unit 311 amplifies the echo signal (step S2). Here, the signal amplification unit 311 amplifies the echo signal (STC correction) on the basis of the relationship between the amplification factor and the reception depth illustrated in FIG. 2, for example.

Thereafter, the B-mode image data generation unit 341 generates B-mode image data using the echo signal amplified by the signal amplification unit 311 and outputs this data to the display device 4 (step S3). After receiving the B-mode image data, the display device 4 displays the B-mode image corresponding to the B-mode image data (step S4).

Subsequently, the region-of-interest setting unit 361 sets the region of interest on the basis of the settings entered via the input unit 35 (step S5: region-of-interest setting step).

The amplification correction unit 331 performs amplification correction on the signal outputted from the transceiver 31 so that the amplification factor is constant irrespective of the reception depth (step S6). Here, the amplification correction unit 331 performs amplification correction so as to establish the relationship between amplification factor and reception depth illustrated in FIG. 3, for example.

Thereafter, the frequency analysis unit 332 calculates the frequency spectrum for all sample data groups by performing frequency analysis using FFT computation (step S7: frequency analysis step). FIG. 22 is a flowchart illustrating an overview of processing executed by the frequency analysis unit 332 in step S7. The frequency analysis processing is described in detail hereinbelow with reference to the flowchart illustrated in FIG. 22.

First, the frequency analysis unit 332 sets the counter k that identifies the sound ray to be analyzed to k₀ (step S21).

Then, the frequency analysis unit 332 sets the initial value Z^{(k)}₀ of the data position (corresponding to the reception depth) Z^{(k)}, which is representative of a series of data groups (sample data groups) to be acquired for FFT computation (step S22). For example, FIG. 4 illustrates a case where the eighth data position of the sound ray SRₖ is set as the initial value Z^{(k)}₀, as mentioned earlier.

Thereafter, the frequency analysis unit 332 acquires a sample data group (step S23) and applies the windowing function stored by the storage unit 37 to the acquired sample data group (step S24). By subjecting the sample data group to the windowing function in this way, it is possible to avoid discontinuities in the sample data group at the boundaries and to prevent artifacts from occurring.

The frequency analysis unit 332 then determines whether the sample data group at data position Z^{(k)} is a normal data group (step S25). As described when referring to FIG. 4, the sample data group must have a data count to the power of two. The data count of the normal sample data group is 2ⁿ (n is a positive integer) hereinbelow. In the present embodiment, the data position Z^{(k)} is set, if at all possible, to be the center of the sample data group to which Z^{(k)} belongs. Specifically, since the data count of the sample data group is 2ⁿ, Z^{(k)} is set to the 2ⁿ/2(=2ⁿ⁻¹))th position near the center of that sample data group. In this case, the sample data group is normal, which means that there are 2ⁿ⁻¹−1(=N) data in front of the data position Z^{(k)}, and 2ⁿ⁻¹(=M) data behind the data position Z^{(k)}. In the case illustrated in FIG. 4, the sample data groups F₁, F₂, F₃, . . . , F_{K-1} are all normal. Note that FIG. 4 illustrates a case where n=4 (N=7, M=8).

When, as a result of the determination in step S25, the sample data group at data position Z^{(k)} is normal (step S25: Yes), the frequency analysis unit 332 moves to step S27, which will be described subsequently.

When, as a result of the determination in step S25, the sample data group at data position Z^{(k)} is not normal (step S25: No), the frequency analysis unit 332 generates a normal sample data group by inserting zero data equivalent to the missing amount (step S26). For the sample data group that is determined to be not normal in step S25 (for example, the sample data group F_{K} in FIG. 4), the windowing function is applied before adding the zero data. Therefore, even when zero data is inserted into the sample data group, no data discontinuity will occur. After step S26, the frequency analysis unit 332 moves to step S27, which is described below.

In step S27, the frequency analysis unit 332 performs FFT computation using the sample data group, thus obtaining the frequency spectrum, which is the frequency distribution of the amplitude (step S27).

Then, the frequency analysis unit 332 changes the data position Z^{(k)} by the step width D (step S28). The step width D is assumed to be stored in advance by the storage unit 37. FIG. 4 illustrates a case where D=15. The step width D preferably matches the data step width used by the B-mode image data generation unit 341 when generating B-mode image data. However, when a reduction in the amount of computation in the frequency analysis unit 332 is desired, a value larger than the data step width may also be set as the step width D.

Thereafter, the frequency analysis unit 332 determines whether the data position Z^{(k)} is greater than the maximum value Z^{(k)}ₘₐₓ in the sound ray SRₖ (step S29). When the data position Z^{(k)} is greater than the maximum value Z^{(k)}ₘₐₓ (step S29: Yes), the frequency analysis unit 332 increases the counter k by 1 (step S30). This means that the process is moved to the next sound ray. On the other hand, when the data position Z^{(k)} is less than or equal to the maximum value Z^{(k)}ₘₐₓ (step S29: No), the frequency analysis unit 332 returns to step S23. In this way, the frequency analysis unit 332 performs the FFT computation on [(Z^{(k)}ₘₐₓ−Z^{(k)}₀+1)/D+1] sample data groups for the sound ray SRₖ. Here, [X] is the largest integer not exceeding X.

After step S30, the frequency analysis unit 332 determines whether the counter k is greater than the maximum value kₘₐₓ (step S31). If the counter k is greater than the maximum value kₘₐₓ (step S31: Yes), the frequency analysis unit 332 ends the series of frequency analysis processes. On the other hand, when the counter k is equal to or less than the maximum value kₘₐₓ (step S31: No), the frequency analysis unit 332 returns to step S22. This maximum value kₘₐₓ is a value optionally indicated and inputted by a surgeon or another user via the input unit 35, or is a value which is set beforehand in the storage unit 37.

In this way, the frequency analysis unit 332 performs a plurality of FFT computations for each of the (kₘₐₓ−k₀+1) sound rays in the region to be analyzed. The results of the FFT computations are stored in the storage unit 37 together with the reception depth and the reception direction.

Note that, although it is assumed in the description hereinabove that the frequency analysis unit 332 performs the frequency analysis processing on all areas where ultrasound signals are received, frequency analysis processing can also be performed only in the set region of interest.

Following the frequency analysis processing of step S7 described hereinabove, the feature calculation unit 333 calculates the pre-correction features of the plurality of frequency spectra respectively, and calculates the correction feature of each frequency spectrum by performing attenuation correction to eliminate the effect of ultrasound wave attenuation on the pre-correction feature of each frequency spectrum (steps S8 to S9).

In step S7, the approximation unit 333a calculates the pre-correction feature corresponding to each frequency spectrum by performing regression analysis on the plurality of frequency spectra generated by the frequency analysis unit 332, respectively (step S8). More specifically, the approximation unit 333a approximates each frequency spectrum with a linear equation by means of regression analysis, and calculates the slope a₀, intercept b₀, and midband fit c₀ as the pre-correction feature. For example, the straight line L₁₀ illustrated in FIG. 5 is a regression line that the approximation unit 333a approximates by means of regression analysis of the frequency spectrum C₁ of the frequency band F.

Thereafter, the attenuation correction unit 333b calculates the correction feature by performing attenuation correction using the attenuation factor α on the pre-correction feature approximated for each frequency spectrum by the approximation unit 333a, and stores the calculated correction feature in the storage unit 37 (step S9). The straight line L₁ illustrated in FIG. 6 is an example of a straight line obtained by the attenuation correction unit 333b performing the attenuation correction processing.

In step S9, the attenuation correction unit 333b perform the calculation by substituting the reception depth z in Equations (2) and (4) above with the data position Z=(fₛₚ/2vₛ)Dn obtained using the data array of the sound ray of the ultrasound signal. Here fₛₚ is the data sampling frequency, vₛ is the speed of sound, D is the step width, and n is the number of data steps from the first data of the sound ray to the data position of the sample data group to be processed. For example, if the data sampling frequency fₛₚ is set to 50 MHz, the sound velocity vₛ is set to 1530 m/sec, and the data array illustrated in FIG. 4 is adopted and the step width D is set to 15, then z=0.2295n (mm).

The received echo signal is then used to calculate the relative attenuation factor (step S10). The relative attenuation factor calculation unit 334 calculates the relative attenuation factor for each plane (plane P_{T}1, plane P_{T}2, . . . , plane P_{T}N) in the foregoing flow by comparing the intensity between paths for the region of interest set by the region-of-interest setting unit 361. This step S10 corresponds to the comparison step. The relative attenuation factor calculation unit 334 calculates, for each plane, the relative attenuation factor for which the same region of interest (for example, the region of interest (1,1)) is used as the reference.

In step S11 following step S10, the attenuation-factor evaluation information generation unit 335 generates information for evaluating the attenuation factor according to a preset condition (evaluation information generation step). In this step S11, information for imaging the distribution of relative attenuation factors for a designated plane is generated.

The relative attenuation factor distribution image data generation unit 343 generates relative attenuation factor distribution image data obtained by imaging the distribution of relative attenuation factors, on the basis of the information generated by the attenuation-factor evaluation information generation unit 335 (step S11: relative attenuation factor distribution image data generation step). The relative attenuation factor distribution image data generation unit 343 generates the distribution image data illustrated in FIG. 20, for example.

The feature image data generation unit 342 generates feature image data by superimposing visual information associated with the feature calculated in step S8 and in accordance with the color scheme conditions set in step S12 on each pixel in the B-mode image data generated by the B-mode image data generation unit 341 (step S12: feature image data generation step).

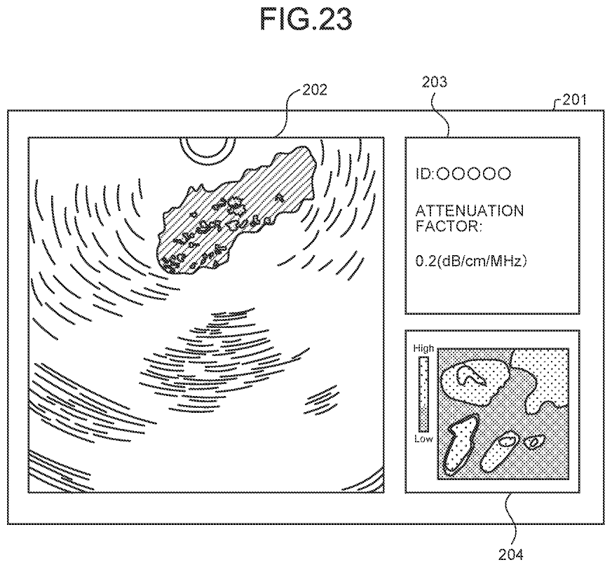

Subsequently, the display device 4 displays the feature image corresponding to the relative attenuation factor distribution image data generated by the relative attenuation factor distribution image data generation unit 343 and/or the feature image data generated by the feature image data generation unit 342 under the control of the control unit 36 (step S13). FIG. 23 is a diagram schematically illustrating a display example of a feature image on the display device 4. The feature image 201 illustrated in FIG. 23 has a superimposed image display unit 202 that displays an image in which visual information relating to the feature is superimposed on the B-mode image, an information display unit 203 that displays identification information, and the like, of the observation target, and a relative attenuation factor information display unit 204 that displays relative attenuation factor information. The relative attenuation factor information display unit 204 may also display statistics instead of a relative attenuation factor distribution image.

The information display unit 203 may also be used to further display information on a feature, approximation formulas, and image information such as gain and contrast, and so forth. Furthermore, the B-mode image corresponding to the feature image may be displayed alongside the feature image, or the B-mode image may be displayed in the superimposed image display unit 202.

In the first embodiment of the disclosure described hereinabove, the relative attenuation factor is calculated using echo signals received via paths through mutually different regions of interest from among a plurality of regions of interest, and a distribution of the relative attenuation factors is generated, and statistics are calculated. By checking the relative attenuation factor, the user is able to accurately evaluate even a test subject with non-uniform reflectance. For example, tissue characteristics can be accurately evaluated by calculating the relative attenuation factor between the reference tissue (a normal liver, for example) for which the attenuation factor value is relatively stable, and the observation target tissue (a pancreatic tumor, for example).

In the foregoing first embodiment, the attenuation correction may also be performed by changing the attenuation factor of each pixel position in a relative manner by using the relative attenuation factor.

Modification Example 1 of First Embodiment

Next, a modification example 1 of the first embodiment of the disclosure will be described. The ultrasound imaging system according to this modification example 1 has the same configuration as the foregoing ultrasound imaging system 1. Processing that is different from that of the first embodiment will be described hereinbelow. In this modification example 1, the region of interest where the reception intensity represents the noise level is excluded from imaging and statistical calculations. In this modification example 1, the region of interest with the noise-level reception intensity is set as a region where the relative attenuation factor cannot be calculated and is excluded from subsequent calculation processing.

According to this modification example 1, by excluding the noise-level region of interest from the calculation target, an image obtained by suppressing the effect of noise when imaging the distribution of relative attenuation factors can be achieved. The statistics calculated from the relative attenuation factor can also be acquired more accurately.

Modification example 2 of first embodiment Next, a modification example 2 of the first embodiment of the disclosure will be described. The ultrasound imaging system according to this modification example 2 has the same configuration as the foregoing ultrasound imaging system 1. Processing that is different from that of the first embodiment will be described hereinbelow. In this modification example 2, the ultrasound transducer 21 transmits a plane wave, and acquires an echo signal corresponding to each region of interest (path) by applying a focus (a delay) upon receipt.

According to this modification example 2, transmitting a plane wave makes it possible to reduce the number of ultrasound wave transmissions, thus improving the frame rate.

Modification Example 3 of First Embodiment

Next, a modification example 3 of the first embodiment of the disclosure will be described. The ultrasound imaging system according to this modification example 3 has the same configuration as the foregoing ultrasound imaging system 1. Processing that is different from that of the first embodiment will be described hereinbelow. In this modification example 3, the relative attenuation factor calculation unit 334 calculates the relative attenuation factor by using the intensity for each frequency (the foregoing intensity I) calculated by the frequency analysis unit 332.

According to this modification example 3, the frequency dependence of the attenuation factor can be evaluated by comparing the relative attenuation factor between frequencies.

### Second Embodiment

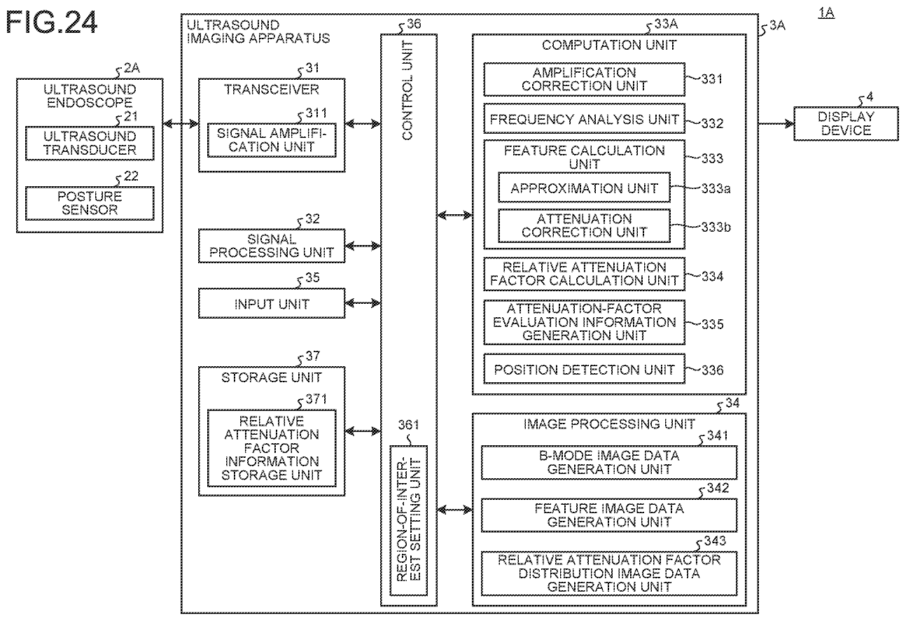

FIG. 24 is a block diagram illustrating the configuration of an ultrasound imaging system 1A provided with an ultrasound imaging apparatus 3A according to a second embodiment of the disclosure. The ultrasound imaging system 1A illustrated in FIG. 24 is provided with an ultrasound endoscope 2 (ultrasound probe) that transmits ultrasound waves to a test subject which is an observation target and receives the ultrasound waves reflected by the test subject, an ultrasound imaging apparatus 3A that generates an ultrasound image on the basis of an ultrasound signal acquired by the ultrasound endoscope 2, and a display device 4 that displays the ultrasound images generated by the ultrasound imaging apparatus 3A. The ultrasound imaging system 1A according to this second embodiment has the same configuration as the foregoing ultrasound imaging system 1, except that the ultrasound endoscope 2 has been changed to an ultrasound endoscope 2A and the ultrasound imaging apparatus 3 has been changed to an ultrasound imaging apparatus 3A. The ultrasound imaging apparatus 3A, which has a different configuration from that of the first embodiment, is described hereinbelow.

The ultrasound endoscope 2A is provided with a posture sensor 22 in addition to the configuration of the foregoing ultrasound endoscope 2. Any known posture sensor (e.g. gyro sensor or acceleration sensor) can be used as the posture sensor 22.

The configuration of the ultrasound imaging apparatus 3A is the same as that of the foregoing ultrasound imaging apparatus 3, except that the computation unit 33 has been replaced with the computation unit 33A. In addition to the configuration of the foregoing computation unit 33, the computation unit 33A is provided with a position detection unit 336. The position detection unit 336, which has a different configuration from that of the foregoing first embodiment, and processing thereof, will be described hereinbelow.

The position detection unit 336 acquires the detection results of the posture sensor and detects the posture of the ultrasound transducer 21. The position detection unit 336 detects the scanned position on the basis of the posture of the ultrasound transducer 21.

The relative attenuation factor calculation unit 334 calculates, by means of the calculation processing described in the first embodiment above, the relative attenuation factor of the planes P_{T}1, P_{T}2, . . . , and P_{T}N (see FIG. 18) for each posture by using the echo signals received by the ultrasound transducer 21 in mutually different postures. At such time, the planes P_{T}1, P_{T}2, . . . , and P_{T}N of each posture are non-parallel to each other and intersect one another.

The attenuation-factor evaluation information generation unit 335 generates information that associates the relative attenuation factors of each posture calculated by the relative attenuation factor calculation unit 334, on the basis of the positions detected by the position detection unit 336. By calculating the relative values between the corresponding relative attenuation factors between postures (for example, the relative attenuation factors at the positions where the planes intersect), a distribution of relative attenuation factors using the same reference can be generated in three-dimensional space.

In this second embodiment, relative attenuation factors in three-dimensional space are generated using relative attenuation factors calculated from echo signals obtained by means of mutually different postures. According to this second embodiment, attenuation factors can be evaluated in relative terms in three-dimensional space.

Here, in the foregoing first embodiment, an evaluation using planes is not assumed because the planes P_{T}1, P_{T}2, . . . , and P_{T}N on which the relative attenuation factors are displayed form curved surfaces when the ultrasound transducer 21 is a convex transducer or a radial transducer. However, in the second embodiment, the relative attenuation factors of planes can be reconstructed from the distribution of the relative attenuation factors in three dimensions. Therefore, the second embodiment makes it possible to provide cross-sectional information that is easy for the surgeon to understand, irrespective of the shape of the transducer, and this information can also be easily compared with images of other tomographic modalities (CT, MRI, and the like). For example, tissues close to each other (the liver and pancreas, or the like) can be captured using a single two-dimensional image, whereas tissues separate from each other (the liver and rectum, or the like) are difficult to capture using a single two-dimensional image. In such cases, by calculating relative attenuation factors in three dimensions as per the second embodiment, it is possible to calculate the relative attenuation factor between tissues that are spatially separated, thereby enabling even separate tissues to be compared.

Although modes for carrying out the disclosure have been described thus far, the disclosure should not be limited only by the foregoing embodiments. The disclosure may include various embodiments and so forth which have not been disclosed here. In the foregoing first and second embodiments, an extracorporeal ultrasound probe, which emits ultrasound waves from the body surface of the test subject, may also be applied as an ultrasound probe. Extracorporeal ultrasound probes are usually used when observing abdominal organs (liver, gallbladder, bladder), breasts (especially mammary glands), and the thyroid gland.

In addition, although the foregoing first and second embodiments are described as having a configuration in which a feature is calculated by performing frequency analysis, an embodiment is also possible which does not have a configuration for calculating a feature, that is, a configuration that does not include the frequency analysis unit 332, the feature calculation unit 333, or the feature image data generation unit 342.

Moreover, in the foregoing first and second embodiments, the disclosure was described as being provided with the ultrasound transducer 21 which has a plurality of two-dimensionally arranged piezoelectric elements. However, the configuration may also be such that piezoelectric elements are arranged in a one-dimensional (linear) manner.

As described hereinabove, the ultrasound imaging apparatus, the operating method of the ultrasound imaging apparatus, and the operating program for the ultrasound imaging apparatus according to the disclosure are useful for accurately evaluating even a test subject with non-uniform reflectance.

The disclosure affords the advantageous effect of enabling an accurate evaluation even in the case of a test subject of nonuniform reflectance.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound imaging apparatus, comprising:
a processor configured to
transmit a signal for transmitting an ultrasound wave toward an observation point from an ultrasound probe,
receive an echo signal that is obtained by converting an ultrasound wave received by the ultrasound probe into an electrical signal,
generate information relating to an attenuation factor by comparing a first intensity of a first echo signal with a second intensity of a second echo signal, the first echo signal being a signal which, after being transmitted via a first path and being reflected by the observation point, has been received via the first path, the second echo signal being a signal which, after being reflected by the observation point, has been received via a second path, the second path being a path that is different from the first path and that is equal in length to the first path, and
generate evaluation information representing a comparison result.

2. The ultrasound imaging apparatus according to claim 1,
wherein the processor is further configured to set a first region of interest and a second region of interest for data groups of scanning planes of the ultrasound probe, and
wherein the first region of interest includes the first path, and the second region of interest includes the second path.

3. The ultrasound imaging apparatus according to claim 2,
wherein the processor is further configured to calculate a relative attenuation factor by taking a differential of an attenuation factor of the second region of interest relative to an attenuation factor of the first region of interest.

4. The ultrasound imaging apparatus according to claim 2,
wherein the processor is further configured to generate the evaluation information for evaluating the attenuation factor.

5. The ultrasound imaging apparatus according to claim 3,
wherein the processor is further configured to
receive the echo signal from the ultrasound probe in which a plurality of elements for transmitting and receiving the ultrasound wave are arranged two-dimensionally, and
when a plurality of planes are at mutually different distances from a transmission-reception plane for the ultrasound wave in the ultrasound probe and each of the planes is a set of points which are equidistant from the transmission-reception plane, calculate the relative attenuation factor of each plane among the plurality of planes.

6. The ultrasound imaging apparatus according to claim 2,
wherein the processor is further configured to exclude, from a comparison, a region of interest for which an intensity of the echo signal is equal to or greater than a threshold value representing noise.

7. The ultrasound imaging apparatus according to claim 1,
wherein the processor is further configured to cause the ultrasound probe to transmit a plane wave and to receive the ultrasound wave with a delay.

8. The ultrasound imaging apparatus according to claim 1,
wherein the processor is further configured to
perform frequency analysis by applying a fast Fourier transform based on the echo signal, thereby calculating a frequency spectrum,
implement wide-area pulse transmission with respect to the ultrasound probe, and
compare an intensity of the echo signal for each frequency based on the calculated frequency spectrum.

9. The ultrasound imaging apparatus according to claim 5, further comprising:
a position detector configured to detect a position of a scanning plane by detecting a posture of the ultrasound probe,
wherein the processor is further configured to associate the calculated relative attenuation factor with the position to generate three-dimensional spatial information for the calculated relative attenuation factor.

10. An operating method of an ultrasound imaging apparatus configured to generate an ultrasound image based on an ultrasound signal acquired by an ultrasound probe provided with an ultrasound transducer that transmits an ultrasound wave toward an observation target and receives an ultrasound wave reflected by the observation target, the method comprising:
transmitting a signal for transmitting an ultrasound wave toward an observation point from the ultrasound probe,
receiving an echo signal that is obtained by converting an ultrasound wave received by the ultrasound probe into an electrical signal;
generating information relating to an attenuation factor by comparing a first intensity of a first echo signal with a second intensity of a second echo signal, the first echo signal being a signal which, after being transmitted via a first path and being reflected by the observation point, has been received via the first path, the second echo signal being a signal which after being reflected by the observation point, has been received via a second path, the second path being a path that is different from the first path and that is equal in length to the first path; and
generating evaluation information representing a comparison result.

11. A non-transitory computer-readable recording medium with an executable program stored thereon, the program causing an ultrasound imaging apparatus configured to generate an ultrasound image based on an ultrasound signal acquired by an ultrasound probe provided with an ultrasound transducer that transmits an ultrasound wave toward an observation target and receives an ultrasound wave reflected by the observation target, to execute:
transmitting a signal for transmitting an ultrasound wave toward an observation point from the ultrasound probe,
receiving an echo signal that is obtained by converting an ultrasound wave received by the ultrasound probe into an electrical signal;
generating information relating to an attenuation factor by comparing a first intensity of a first echo signal with a second intensity of a second echo signal, the first echo signal being a signal which, after being transmitted via a first path and being reflected by the observation point, has been received via the first path, the second echo signal being a signal which, after being reflected by the observation point, has been received via a second path, the second path being a path that is different from the first path and that is equal in length to the first path; and
generating evaluation information representing a comparison result.

12. An ultrasound imaging apparatus, comprising:
a processor configured to
transmit a signal for transmitting an ultrasound wave toward an observation point from an ultrasound probe,
receive an echo signal that is obtained by converting an ultrasound wave received by the ultrasound probe into an electrical signal,
set a first region of interest including a first path and a second region of interest including a second path for data groups on scanning planes of the ultrasound probe,
compare a first intensity of a first echo signal with a second intensity of a second echo signal, the first echo signal being a signal which, after being transmitted via the first path and being reflected by the observation point, has been received via the first path, the second echo signal which, after being reflected by the observation point, has been received via the second path, the second path being a path that is different from the first path and that is equal in length to the first path, and
generate evaluation information representing a comparison result.
